SEQUENCE LISTING (2 sequences)

SEQ ID NO: 1 - protein, 9 aa, Homo sapiens
CYIQNCPLG

SEQ ID NO: 2 - protein, 9 aa, Homo sapiens
CYIQDCPLG

(19) United States
(12) Patent Application Publication
(10) Pub. No.: US 2006/0252685 A1
(43) Pub. Date: Nov. 9, 2006

(54) TREATMENT FOR SLEEP APNEA

(76) Inventor: Jeffrey D. Gould, Bethlehem, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/381,383

(22) Filed: May 3, 2006

Related U.S. Application Data
(60) Provisional application No. 60/677,727, filed on May 4, 2005.

(51) Int. Cl. A61K 38/12

(52) U.S. Cl. 514/12

(57) ABSTRACT

The present invention relates to compositions and methods of alleviating a symptom of sleep apnea, snoring associated with and independent of sleep apnea, and/or sudden infant death syndrome by administering oxytocin. Specifically, the invention includes a method of administering oxytocin or a salt thereof to an animal in order to treat obstructive sleep apnea, snoring associated with and independent of sleep apnea, and sudden infant death syndrome.

### CROSS REFERENCE TO RELATED APPLICATIONS

This Application is entitled to priority pursuant to 35 U.S.C. 119(e) to U.S. Provisional Patent Application 60/677,727, which was filed on May 4, 2005.

### BACKGROUND OF THE INVENTION

1. Field of the Invention

This invention relates to the treatment of sleep apnea with oxytocin. Specifically, this invention discloses the use of oxytocin to treat obstructive sleep apnea, snoring associated with and independent of sleep apnea, and/or sudden infant death syndrome.

2. Description of Related Art

Obstructive Sleep Apnea (OSA) affects approximately 4 percent of all adult males and 2 percent of all adult women; however, the prevalence of pediatric OSA is unknown. The term “apnea” is defined as a pause in breathing. With OSA, the apnea occurs for ten seconds or more. This disorder involves the subtotal or complete closure of the oropharynx recurrently during sleep. This closure must take place greater than or equal to five times per hour for a diagnosis of OSA. With throat closures, there is an associated oxygen desaturation of the blood recurrently during sleep, snoring, three second or longer awakenings, and witnessed breathing pauses during sleep. This problem usually lasts for decades, possibly starting in childhood.

There is clinical evidence that this process of apnea is linked with increased risks of stroke, myocardial infarction, uncontrollable hypertension, obesity, and excessive anytime sleepiness. There also exists some indirect evidence that OSA is linked with an increased risk of Alzheimer's disease. Treatment of sleep apnea, therefore, is associated with a remediation of throat closure; however, current treatments are usually incompletely effective in the treatment of OSA. The standards of care include the use of positive airway pressure and surgical removal of uvulopalatal tissue. Both are limited treatments because of patient tolerance and completeness of remediation of the problem. A more direct approach would be to prevent the muscle relaxation of the airway instead of attempting to pry the tissues apart with air, by tissue removal or by tracheostomy.

Muscles in the throat are two types, skeletal and smooth. Higher in the throat, the proportion of skeletal muscle to smooth muscle is greatest, reversing at the base of the throat. Muscle tissues of all types are covered with receptors which dictate when constriction and relaxation take place. The receptors are stimulated by nerve impulses which originate from the brain. Smooth muscle comprises both muscarinic and nicotinic receptors; however, it varies by the amount of receptor types depending on where it is located. Chemical interactions routinely occur changing the degree of relaxation of the smooth muscle without conscious control.

Skeletal muscle is under direct mental control allowing for functions such a phonation and voluntary swallowing. REM sleep is an example of complete atonia of the skeletal muscle. Smooth muscle also has a tone reduction in sleep which worsens in REM sleep. Of note is that this stage of sleep is typically when OSA is at its worst. If muscarinic receptors can be stimulated on smooth muscle receptors in the throat during sleep, the muscles would remain contracted. These contracted muscles would keep the airway forced open remediating apnea which is caused by recurrent throat closure.

Oxytocin is a naturally produced hormone from a brain structure called the pituitary gland and has been reported as comprising the amino acid sequence of CYS-TYR-ILE-GLN-ASN-CYS-PRO-LEU-GLY (SEQ ID NO: 1) or CYS-TYR-ILE-GLN-ASP-CYS-PRO-LEU-GLY (SEQ ID NO: 2). Oxytocin is known to interact with the smooth muscle of the uterus. Specifically, it binds to muscarinic receptors which produce contraction of uterine muscle. The muscarinic smooth muscle receptors of the uterus are the same as in other body organs including the throat. Oxytocin, which is commercially produced by Pfizer Inc. (New York, N.Y.) is available as an intramuscular, intravascular, and/or intranasal medication used to induce and increase uterine muscle contractions to facilitate childbirth. Thus, currently the medication is only utilized when there are inadequate uterine contractions. Oxytocin also stops and prevents excessive uterine hemorrhage. Side and/or adverse effects with this medication are almost unknown from the tens of thousands of uses over the course of numerous years; however, anaphylaxis, cardiac arrhythmias, and the syndrome of inappropriate ADH secretion (SIADH) are possible.

Contraction of the throat which is inadequate in sleep apnea, particularly OSA, snoring associated with and independent of sleep apnea, and/or sudden infant death syndrome are remediated by a steady dose of oxytocin. Oxytocin produces an increased smooth muscle tone in the throat leading to prevention of closure of the throat. By increasing oropharyngeal smooth muscle tone as it does for the uterus, exogenous oxytocin adequately treats OSA in both genders, adults, as well as the pediatric population without significant side-effects, snoring associated with and independent of sleep apnea, and/or sudden infant death syndrome.

Thus, this invention provides compositions and methods for alleviating a symptom of OSA. snoring associated with and independent of sleep apnea, and/or sudden infant death syndrome by administering oxytocin.

These and other advantages of the present invention, as well as additional inventive features, will be apparent from the description of the invention provided herein.

All references cited herein are incorporated by reference in their entireties.

### BRIEF SUMMARY OF THE INVENTION

In one aspect, the invention provides a method of alleviating at least one symptom of sleep apnea, snoring associated with and independent of sleep apnea, and/or sudden infant death syndrome, the method comprising administering oxytocin or a salt thereof in a therapeutically effective amount. In one embodiment, the sleep apnea is OSA. In another embodiment, the symptom is a symptom selected from the group consisting of relaxation of the throat muscles, snoring, sleepiness, fatigue, lack of energy, lack of coordination, poor concentration, and morning headaches. In a further embodiment, the oxytocin is administered intravenously, intramuscularly, intracutaneously, systemically, nasally, or orally. In a preferred embodiment, the concentration of the oxytocin is from about 0.001 to about 30 units/minute. In one embodiment, the oxytocin is administered daily or at the time of the sleep apnea. In yet another embodiment, the oxytocin is administered weekly, biweekly or monthly. In a further embodiment, the oxytocin is administered to an adult, child or infant.

In another aspect, the invention includes a composition comprising an oxytocin or a salt thereof and at least one agent selected from the group consisting of an antihistamine, an analgesic, nasal steroid and an anti-inflammatory, wherein the oxytocin or a salt thereof is present in an amount effective to alleviate at least one symptom of sleep apnea in an animal. In one embodiment, the composition further comprises a pharmaceutical carrier selected from the group consisting of water, Ringer's solutions, dextrose solution, 5% human serum albumin and liposomes. In another embodiment, the composition is in the form of a kit and further comprises instructions for use.

### DETAILED DESCRIPTION OF THE INVENTION

This invention relates to the treatment of sleep apnea with oxytocin. Specifically, this invention discloses the use of oxytocin to treat OSA, snoring associated with and independent of sleep apnea, and/or sudden infant death syndrome.

In one aspect, the invention includes a method for treating sleep apnea, particularly OSA, snoring associated with and independent of sleep apnea, and/or sudden infant death syndrome by administration of oxytocin.

In another aspect, the invention includes novel compositions comprising oxytocin and at least one agent selected from the group consisting of an analgesic, an antiinflammatory, a nasal steroid and an antihistamine, wherein the oxytocin is present in an amount effective to alleviate at least one symptom of sleep apnea, particularly OSA, snoring associated with and independent of sleep apnea, and/or sudden infant death syndrome in an animal. In one embodiment, the antiinflammatory is a non-steroidal antiinflammatory drug. In another embodiment, the composition further comprises a taste enhancer.

The term “composition” refers to a combination of at least two compounds, for example oxytocin and an analgesic, wherein the compounds may be administered concurrently or sequentially with respect to one another. That is, a first compound (i.e., oxytocin) may be administered prior to, simultaneous with or subsequent to a second compound (i.e., analgesic). The compounds coexist within the animal following administration for at least a minimum amount of time. The invention also contemplates kits comprising the compositions of the invention. The compositions of the invention may also be included in a container, pack, or dispenser together with instructions for administration.

In one embodiment, the oxytocin or composition of the invention is administered parenterally, e.g., intravenously, intradermally, subcutaneously, systemically, orally (e.g., inhalation), intra-orally, transdermally (topical), intramuscularly, vaginally, nasally, transmucosally, and rectally. The amount of the drug, routes of delivery and timing of administration of the drug vary in accordance with the compound employed, the animal species, bodyweight, age and whether the treatment is therapeutic or prophylactic. Accordingly, in most cases dosing and dosages will be carried out according to the manufacturer's instructions or as otherwise known in the art.

Single and multiple dosing regimes are also contemplated in this invention. Multiple dosing regimes may comprise administration of two or more doses to different sites on or by different routes of administration to an animal at the same time. In one embodiment, multiple dosing regimes may comprise the administration of two or more doses of oxytocin or composition of the invention to an animal over a period of time covering hours, days and weeks. In another embodiment, the oxytocin or composition of the invention or composition of the invention is delivered daily. In another embodiment, the oxytocin or composition of the invention is delivered twice daily. In a further embodiment, the oxytocin or composition of the invention is delivered for a period of time until symptoms subside. The amount of drug in a composition may vary within a broad range, as long as effectiveness is maintained. In one embodiment, the oxytocin is administered to a patient at a concentration between about 0.001 to about 30 units/minute. In another embodiment, the oxytocin is administered to a patient at a concentration between about 0.001 to 40 units/minute.

In another embodiment, the oxytocin or composition of the invention is administered to an animal. In a preferred embodiment, the animal is a mammal. In a more preferred embodiment, the mammal is a human.

The compositions of the invention can be incorporated into pharmaceutical compositions suitable for administration. Such compositions typically comprise the drug(s) and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except in so far as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. As stated above, examples of routes of administration include parenteral, e.g., intravenous, intradermal, subcutaneous, systemically, oral (e.g., inhalation), intra-oral, transdermal (topical), intramuscular, vaginal, nasal, transmucosal, and rectal administration. In one embodiment, the oxytocin is administered orally. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (i.e., oxytocin) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. In one embodiment, a taste enhancer is used with the oral oxytocin composition. For the purpose of oral therapeutic administration, the active compound can be incorporated with excipients and used in the form of tablets, spansules, troches, or capsules. Oral compositions can also be prepared using a fluid carrier for use as a mouthwash, wherein the compound in the fluid carrier is applied orally and swished and expectorated or swallowed. Pharmaceutically compatible binding agents. and/or adjuvant materials can be included as part of the composition. The tablets, pills, spansules, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral, nasal or parenteral compositions in dosage unit form for ease of administration and uniformity of dosage. Dosage unit form as used herein refers to physically discrete units suited as unitary dosages for the subject to be treated: each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The invention will be illustrated in more detail with reference to the following Examples, but it should be understood that the present invention is not deemed to be limited thereto.

### EXAMPLES

### Example 1

### Treatment of OSA, Snoring Associated with and Independent of Sleep Apnea, and/or Sudden Infant Death Syndrome with Oxytocin

Oxytocin, a medication produced to increase uterine muscle contraction, is administered in intravenous and/or intramuscular form to patients previously diagnosed with OSA. The treatment leads to contraction of smooth muscle in the throat which then forces open a closed throat treating the cause of OSA, snoring associated with and independent of sleep apnea, and/or sudden infant death syndrome.

The oxytocin is delivered in a steady state by intravenous infusion at 0.0001 units/minute, but the best dosing is 10 units intramuscular at the time of sleep. Pediatric formulation is dosed using a fraction of 10 units intramuscular based on body weight (10 units for 70 kg person, 5 units for a 35 kg person, etc.). Subcutaneous, nasal spray, suppository, and melt-tab use are also acceptable at a delivery rate of 0.001 units/minute or 10 units delivered over a 7 hour period. The injection does not have to be local to the orophparynx but instead represents a systemic response to a distant injection. An extended release PO form of the medication also has the same effect for the entire sleep period. The molecule, as commercially available, is utilized to produce this treatment of OSA.

What is claimed is:

1. A method of alleviating at least one symptom of sleep apnea, snoring associated with and independent of sleep apnea, and/or sudden infant death syndrome, the method comprising administering oxytocin or a salt thereof in a therapeutically effective amount.

2. The method of claim 1, wherein the sleep apnea is obstructive sleep apnea.

3. The method of claim 1, wherein the symptom is a symptom selected from the group consisting of relaxation of the throat muscles, snoring, sleepiness, fatigue, lack of energy, lack of coordination, poor concentration, and morning headaches.

4. The method of claim 1, wherein the oxytocin is administered intravenously, intramuscularly, intracutaneously, systemically, nasally, or orally.

5. The method of claim 1, wherein the concentration of the oxytocin is from about 0.001 to about 30 units/minute.

6. The method of claim 1, wherein the oxytocin is administered daily or at the time of the sleep apnea.

7. The method of claim 1, wherein the oxytocin is administered weekly, biweekly or monthly.

8. The method of claim 1, wherein the oxytocin is administered to an adult, child, or infant.

9. A composition comprising an oxytocin or a salt thereof and at least one agent selected from the group consisting of an antihistamine, an analgesic, nasal steroid and an antiinflammatory, wherein the oxytocin or a salt thereof is present in an amount effective to alleviate at least one symptom of sleep apnea in an animal.

10. The composition of claim 9 further comprising a pharmaceutical carrier selected from the group consisting of water, Ringer's solutions, dextrose solution, 5% human serum albumin, and liposomes.

11. The composition of claim 9, wherein the composition is in the form of a kit and further comprises instructions for use.
